(19) United States
(12) Patent Application Publication
(10) Pub. No.: US 2004/0077590 A1
(43) Pub. Date: Apr. 22, 2004

(54) Use of soluble monovalent oligosaccharides as inhibitors of HIV-1 fusion and replication

(76) Inventors: James L. Leach, Columbus, OH (US); Stacey Ann Garber, Dublin, OH (US); Pedro A. Prieto, W. Worthington, OH (US); Balbino Alarcon, Madrid (ES); Ricardo Rueda, Granada (ES); Enrique Vazquez, Granada (ES)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/277,259

(22) Filed: Oct. 22, 2002

(51) Int. Cl. A61K 31/715

(52) U.S. Cl. 514/54

(57) ABSTRACT

The subject invention relates to monovalent oligosaccharides and their use, for example, in the treatment and prevention of mammalian disease caused by HIV-1 virus infections. In particular, the oligosaccharides globotriose and lactose may be used alone or in combination to competitively inhibit the formation of the viral fusion complex that occurs upon infection.

### TECHNICAL FIELD

The invention relates generally to compositions containing monovalent oligosaccharides, specifically lactose and globotriose, and to uses thereof. In particular, such compositions have the ability to inhibit HIV-1 infectivity and syncytia formation. Specifically, globotriose and lactose can be used to competitively inhibit formation of the viral fusion complex.

### BACKGROUND OF THE INVENTION

Human Imunodeficiency Virus (HIV) is a retrovirus that causes Acquired Immune Deficiency Syndrome, AIDS. HIV primarily infects cells with CD4 cell-surface receptor molecules, using them to gain entry. It is well established that many of the molecules involved in HIV cell entrance are the keys to future HIV disease treatment and prevention.

Virtually all AIDS cases in United States and Europe are associated with HIV-1 infection. According to the use of cellular coreceptors, there are two different types of HIV-1 strains: the T-tropic—strains that infect preferentially T cells and form syncytia—and the M-tropic—strains that infect preferentially macrophages and do not form syncytia. T-tropic strains use preferentially the chemokine receptor CXCR4 (in addition to CD4) for entering cells. M-tropic strains preferentially use the chemokine receptor CCR5 (in addition to CD4) for entering cells. HIV-2, which was identified years later from West African AIDS patients, is only partially homologous to HIV-1 and genetically more closely related to the Simian Immunodeficiency Virus (SIV) than HIV-1. The transmission of HIV-1 and HIV-2 are similar; however, HIV-2 transmits less efficiently, particularly via heterosexual and perinatal modes. Furthermore, the mortality rate from HIV-2 infection is only two-thirds that for HIV-1.

The complexity of HIV-1 is evidenced by the vast number of proteins encoded by the virus that give rise to a wide variety of pathogenic mechanisms to sustain infection and resist natural and pharmaceutical defenses. This complexity emphasizes the importance of intervening early in viral transmission and cell entry in order to prevent infection and/or the development of AIDS.

HIV-1 enters permissive cells (CD4′) by binding to CD4 receptors on the target cell's surface. The fusion of HIV-1 requires the formation of a tri-molecular complex between the viral protein gp120, CD4 and the appropriate chemokine receptor, either CXCR4 or CCR5 (E. A. Berger, et al., Ann. Rev. Immunol., 17:657-700, 1999). Recent research has demonstrated that gp120 also specifically interacts with some glycosphingolipids (D. Hammache, et al., J. Biol. Chem. 273:7967-7971, 1998).

Glycosphingolipids are ubiquitous membrane components of the plasma membrane. All share a common hydrophobic transmembrane skeleton that consists of a fatty acid chain and a sphingosine base. This common structure is bound to a variable hydrophilic oligosaccharide residue that protrudes out to the extracellular space. Glycosphingolipids are classified into three main series, ganglioseries, globoseries and lactoseries, according to their carbohydrate structure (S-I. Hakomori and Y. Igarashi, J. Biochem., 118:1091-1103, 1995).

Cumulative evidence indicates that, in addition to protein coreceptors, HIV-1 uses cell surface glycosphingolipids, specifically Gb3 and GM3, as cofactors for fusion (D. Hammache et al., J. Virol. 73:5244-5248, 1999). The neutral glycosphingolipid Gb3, or globotriaosylceramide, has a globotriose saccharide head. The acidic glycosphingolipid GM3 has a 3′sialyllactose head. Recent findings have shown that 1) depletion of complex glycoshpingolipids from the membrane of normally fusion-competent target cells renders them impervious to HIV-1 fusion, and further that 2) the addition of Gb3 specifically can rescue the fusion event in these depleted target cells, indicating that Gb3 is required for efficient viral fusion (A. Puri et al., Proc. Natl. Acad. Sci. 95:14435-14440, 1998; A. Puri et al., Biochem. Biophys. Res. Comm. 242:219-225, 1998). Other research has demonstrated the involvement of GM3 in the fusion event, although the ability of GM3 in restoring the fusion activity of HIV-1 appears to be lower than that reported for Gb3 (P. Hug et al., J. Virol., 74: 6377-6385, 2000).

Oligosaccharides conjugated to a variety of chemical backbones in such a way as to produce a polyvalent oligosaccharide presentation (multiple globotriose moieties at the end of several arms) have been used to treat diseases caused by Shiga and Cholera toxins. The recognition site for these toxins is the oligosaccharide portion of the globoseries-glycolipid globotriaosylceramide, Gb3, known as globotriose (E. A. Merrit and W. G. J. Hol, Curr. Opin. Struct. Biol., 5:165171, 1995). Synthetic oligosaccharide analogues, in the form of multivalent clusters, covalently bound to insoluble silica particles, competitively adsorb the toxins and protect susceptible cells, confirming the potential of carbohydrates as new and viable anti-adhesive therapeutic tools (P. I. Kilov et al., Nature, 403:669-672, 2000). However, present technology relies only upon a multivalent presentation of Gb3, not on monovalent free oligosaccharides.

Infection by HIV starts after initial entry of HIV into the cells. HIV is primarily spread as a sexually transmissible disease. HIV can be transmitted also by parenteral exposure, which is the most efficient method of transmission, close to 90%. HIV infection can also be acquired as a congenital infection perinatally or in infancy. Neonates acquire HIV infections mainly through vertical transmission; mothers with HIV infection can pass the virus transplacentally, at the time of delivery through the birth canal or through breast milk.

The progression of HIV infection into clinical stages is marked by the appearance of typical opportunistic infections or neoplasms, and by the appearance of syncytia-forming variants of HIV. These syncytia-forming variants, derived from non-syncytia-forming variants have greater CD4 cell tropism and are associated with more rapid CD4′ decline. The syncytia-forming variants arise prior to the onset of clinical AIDS, however, appearance of syncytia-forming variants of HIV is a marker for progression of AIDS.

Attempts to cure AIDS have not been met with a high degree of success but have been successful in the management of this viral infection. The strategies to manage AIDS include inhibition of reverse transcriptase with AZT and several strategies directed to the inhibition of the fusion/entry mediated through the use of blocking agents for the CD4 and chemokine receptors, or through the genetic depletion of said receptors (E. A. Berger et al., Ann. Rev. Immunol. 17:657-700, 1999). Compositions containing modified proteins capable of binding to CD4 receptors have been also described as potential tools to competitively prevent binding of HIV-1 and therefore HIV-1 infection (E. A. Berger et al., Ann. Rev. Immunol. 17:657-700, 1999; U.S. Pat. No. 5,985,275). However, most of these drugs are very expensive and undesired effects on the host have not been fully assessed, especially in vertical HIV transmission cases. Prophylaxis with anti-HIV agents and caesarean section before labor have reduced only slightly the risk of vertical HIV transmission (Grosch-Worner et al., AIDS 14:29-3-2911, 2000). In addition, the mutation rate and emergence of resistant human immunodeficiency viruses is very significant in regular drug therapies.

In view of the above, there is an urgent need to expand the diversity of compounds with potential biological activity directed to preventing HIV-1 and other syncytia-forming viruses from infecting host cells. Polyvalent oligosaccharides that interfere with the binding of glycosphingolipids or their synthesis can be useful in the prevention or treatment of HIV infection (WO 00/29556) through the same mechanism as they have been effective in preventing several pathogen driven interactions with cell oligosaccharides. However, monovalent oligosaccharides have been never considered as potential tools for preventing or treating HIV-1 infections. The monovalent oligosaccharides of the present invention have been shown to prevent these interactions. The monovalent oligosaccharides in fact prevented fusion of HIV-1 virus with target human cells, thereby preventing replication of the virus in question. Furthermore, these oligosaccharides can be produced in large quantities at a reasonable cost (U.S. Pat. No. 5,945,314) rather than producing complex multivalent carbohydrate compositions.

All U.S. patents and publications referred to herein are hereby incorporated in their entirety by reference.

### SUMMARY OF THE INVENTION

The present invention relates to a method of using monovalent soluble oligosaccharide decoys, specifically globotriose (the saccharide portion of Gb3) and lactose (the saccharide portion of GM3), to competitively inhibit HIV-1 fusion with cell membranes.

Furthermore, the present invention relates to a method of using monovalent soluble oligosaccharide decoys to prevent the formation of syncytia resulting after infection with HIV-1.

Moreover, the present invention relates to a method of preventing infections caused by HIV-1 by administering the monovalent soluble oligosaccharides alone or in combination. The present invention relates also to a method of treating established retroviral infections by administering soluble oligosaccharides alone or in combination.

Also, the present invention relates to a composition comprising at least one monovalent oligosaccharide or, a combination of at least two monovalent oligosaccharides. The monovalent saccharide may be, for example, globotriose or lactose.

The composition of the present invention possesses a number of advantages over prior anti-HIV-preparations. First, the monovalent oligosaccharides can be combined with a resulting synergistic effect that requires the use of lower concentrations of each of the oligosaccharides than when used alone. Second, globotriose, lactose and other relevant oligosaccharide decoys can be synthesized in large quantities for a practical cost. Third, the composition can be administered at high doses intravenously, orally or dermally (as a lubricant or spermicidal to be used pre- or post-coitus) with little to no toxic effects. Fourth, globotriose and lactose can specifically inhibit an early event required for infection by the HIV-1 virus. As such, the present invention represents a means to prevent infection rather than a post-infection tool. The invention is unique among other therapeutic approaches to HIV-1 with respect to this property among others. Fifth, globotriose and lactose may be conjugated to other chemical moieties to enhance oral absorption and therapeutic half-life.

Additionally, the composition of the present invention can be used in several ways, including (a) as a pharmaceutical agent administered intravenously, orally or dermally, (b) conjugated to an acceptable chemical moiety to enhance oral absorption, (c) as an ingredient in foods or food supplements to be consumed orally via feeding or parenterally.

### BRIEF DESCRIPTION OF THE DRAWINGS

FIG. 1 illustrates the effect of selected oligosaccharides on HIV-1 replication in MT2 cells, A (lacto-N-tetraose), B (globotriose) and C (lactose) at concentrations ranging between 0.5-50 mM. Viral antigen p24 (Ag24) release was measured as an estimate of viral replication (experiment 1).

FIG. 2 shows the effects of selected oligosaccharides on HIV-1 replication in MT2 cells (experiment 2).

FIG. 3 illustrates the effects of oligosaccharides on the formation of syncytia in MT2 cells. Formation of syncytia was analyzed by phase contrast microscopy and release of p24 to the culture supernatant was also recorded.

FIG. 4 illustrates the effect of selected oligosaccharides on CD4 expression in MT2 cells. CD4 expression was evaluated by flow cytometry at 3 and 5 days post-infection.

FIG. 5 shows the effect of selected oligosaccharides on HIV-1 replication in monocytes. Oligosaccharides, A (lacto-N-tetraose), B (globotriose) and C (lactose) were used at concentrations ranging between 0.5-50 mM. Viral antigen p24 (Ag24) release was measured as an estimate of viral replication.

FIG. 6 illustrates the synergistic effect of combined oligosaccharides on HIV-1 replication. 25 mM globotriose (B) and 5 mM lactose (C) were added in different combinations. Fold inhibition (bottom panel) was calculated by dividing p24 release in the absence of drugs by each experimental point.

### DETAILED DESCRIPTION OF THE INVENTION

As noted above, the subject matter of the present invention relates to compositions comprising at least one monovalent oligosaccharide and a pharmaceutically acceptable carrier, which can be used to prevent or treat infections caused by HIV-1 virus.

Definitions and Related Information

As used herein, the term “monovalent” means a single chemical unit with a free anomeric carbon which is not conjugated or bound to an inert matrix and which lacks a synthetic linking arm. An “oligosaccharide” is a sugar molecule that contains approximately 2-10 sugar units. The sugar units (i.e., (CH₂O)n) in an oligosaccharide are connected by glycosidic linkages. Examples of the monovalent oligosaccharides of the present invention which may be used in the treatment and prevention of, for example, HIV-1 infection include, for example, the trisaccharide globotriose (also known as galactose α1-4galactose β1-4glucose) and the disaccharide lactose (also known as o-β-D-galactopyranosyl-(1-4)-β-D-glucopyranose). It should be noted, however, that the use of any monovalent oligosaccharide that has the ability to competitively inhibit binding of viral gp120 to the cellular target(s) is considered to fall within the scope of the present invention. Such oligosaccharides are all readily soluble.

As used herein, a “retrovirus” is a virus that has RNA as its genome but needs to transcribe it to DNA during its replicative cycle within the infected cell. When a retrovirus infects a cell, it must use its reverse transcriptase enzyme to transcribe its RNA to host cell proviral DNA. This DNA becomes integrated in the host chromosomes, and it is this proviral DNA that directs the cell to produce additional virions that are released subsequently. Retroviruses in general contain three major genes: Gag, Pol, and Env. The major structural components coded by Env include the outer envelope glycoprotein gp120 and the transmembrane glycoprotein gp41 derived from glycoprotein precursor gp160. In the case of HIV-1, the virus enters cells by binding to the cellular CD4 receptor, followed by gp120-gp41-mediated fusion of the viral and target cell membranes.

As used herein, “CD4⁺ cells” means cells expressing the CD4 receptor on the cell surface. CD4 is the primary receptor for the binding of the viral outer envelope glycoprotein, Env. After binding, fusion of viral envelope and CD4⁺ cells occurs, leading to entry of the viral particle into the cell.

“Chemokine receptors” as used herein, means coreceptors required for HIV-1 entry. This notion resulted from the fact that CD4 expression was not sufficient to explain HIV-1 entry in target cells. The chemokine receptors most frequently used as HIV-1 receptors are CXCR4 and CCR5 for T-tropic and M-tropic strains of HIV-1, respectively.

A “syncytia-forming virus” is a virus that, after infecting susceptible cell cultures, produces cytopathogenic effects in the form of syncytia. HIV-1, for example, is a syncytia-forming virus.

“Syncytia” or “giant cells” are large masses containing up to 100 nuclei and are believed to result from the fusion of virus-infected cells with non-infected cells. The formation of syncytia may be analyzed, for example, by phase contrast microscopy using specific staining, for the visualization of nuclei.

“MT2 cell” is a human T lymphotropic virus-transformed cell expressing the CD4 receptor.

As used herein, the term “T-tropic” refers to HIV-1 isolates that show efficient infectivity for continuous CD⁴⁺ T cell lines, but poor infectivity for macrophages. This phenotype was originally observed with isolates that had been produced in the laboratory (X4 and pNL strains) and are generally syncytia-forming strains.

Another indicator of the progression of HIV infection, in addition to the formation of syncytia, is the presence of p24 antigen. Although only about 60% of HIV-infected persons develop p24 antigenemia prior to the onset of clinical AIDS, the p24 antigen is a highly specific predictor of the progression of HIV infection both in vitro and in infected patients. Viral replication in cells is followed by the measurement of HIV p24 antigen using an antigen capture immunoassay a decreased signal is an indication of a retarded or decreased release of p24. Said decrease in the presence of oligosaccharides is an indication of the inhibitory effect of the oligosaccharides on viral replication.

A “clinical T-tropic isolate” such as isolate 1936 (Mufioz-Fernandez et al, 1996) is a T-tropic virus obtained from infected donors and maintained in primary cells such as human T lymphoblasts.

The term “M-tropic” refers to HIV-1 isolates (such as the Ba-L strain, [Gartner et al, 1986]) that are nonsyncytia-forming and that infect primarily macrophages.

Cells are infected at different “MOI”, which means multiplicity of infection. In particular, the MOI is the number of virus particles or infectious units adsorbed per cell.

In terms of mechanism of action, the antiviral activity of the monovalent oligosaccharides of the present invention is not mediated by a down regulation or masking of the CD4 or chemokine receptors. The monovalent oligosaccharides of the present invention are thought to act as decoys and prevent the binding of the viral protein gp120 to the cellular target, thereby preventing HIV-1 fusion with the target cell. Specifically, the unconjugated monovalent oligosaccharides of the present invention may competitively inhibit the viral fusion by serving as alternative receptors for the viral gp120, as opposed to the oligosaccharide heads of the glycosphingolipid surface on the target cells.

Therefore, the monovalent oligosaccharides of the present invention present several advantages over other existing approaches. First, the antiviral activity of the unconjugated monovalent oligosaccharides is effective not only as an important treatment tool, but also as a preventive measure by blocking the initial binding of the viral protein to the cellular target. By administering the oligosaccharides the first contact of the viruses with susceptible cells will not occur, preventing the entrance and further replication of virions. Further, it should be noted that the monovalent oligosaccharides described herein may be used to inhibit not only HIV-1 interactions but also other pathogen-driven interactions, currently inhibited by complex polyvalent carbohydrate compounds, as well.

Second, another advantage of the oligosaccharides of the present invention is the synergy of effects resulting from the combination of globotriose and lactose. When administered together, the inhibitory effect on HIV-1 replication is increased to more than double compared to the effect of each oligosaccharide alone.

Third, with respect to production, the monovalent oligosaccharides of the present invention may be made either recombinantly or synthetically, using techniques known in the art, rather than producing complex polyvalent arrays of oligosaccharides. For example, U.S. Pat. No. 5,945,314, incorporated in its entirety herein by reference, describes a method of synthetically producing oligosaccharides.

Further, in order to treat or prevent HIV-1 infection, including vertical transmission at the time of delivery, the monovalent oligosaccharides of the present invention can be administered to adults, infants or newborns, enterically or parenterally. For example, the oligosaccharide may be utilized in a rehydration or hydration solution provided either orally (e.g., Pedialyte® or Equalyte®, Abbott Laboratories, IL) or intravenously (e.g., saline/D5W).

Additionally, one or more oligosaccharides of the present invention can be utilized in pharmaceutical compositions also comprising a pharmaceutically acceptable carrier. A “pharmaceutically acceptable carrier” is any compatible, non-toxic substance suitable for delivering the oligosaccharide(s) to the patient. Examples include sterile water, alcohol, fats, waxes, inert solids, phosphate buffered saline, oils, or water/oil emulsions. The composition may be in either in the form of a tablet, a capsule, an intravenous liquid or injectable, or a dermal cream. The dosage of the composition as well as the form and method of administration may be readily determined by one of ordinary skill in the art in view of such factors such as age, weight, immune state, etc.

Further, the oligosaccharides may be administered as part of an antibiotic or antiviral “cocktail” comprising several antibiotic agents and/or antiviral agents (e.g. Norvir®, Abbott Laboratories, IL), or in conjunction with other agents being used to treat or prevent the symptoms caused by HIV-1 infection. This is especially important in groups most at risk of infection, through promiscuous sexual activity, drug use and perinatal infection.

General Methods

In general, and as it will be elucidated in detail in the following examples, the mechanism of action of the monovalent oligosaccharides on HIV-1 replication was studied on MT-2 cells infected with HIV-1. Increasing concentrations of oligosaccharides were added to the culture media and their inhibiting effects were compared to controls without oligosaccharides. Viral replication was monitored by measuring viral p24 antigen in the supernatant of the cell culture using an antigen capture immunoassay. Similarly, formation of syncytia was analyzed by phase contrast and fluorescence microscopy with staining to visualize the nucleus.

The inhibitory effects of globotriose and lactose on HIV-1 replication was also tested in monocytes cultures. Similarly to MT-2 cells, viral replication in monocytes was measured by release of viral antigen p24 in controls and in the presence of increasing concentrations of oligosaccharides.

The following examples are offered for illustrative purposes only, and are not intended to limit the scope of the present invention in any way.

### EXAMPLE 1

### Globotriose Prevents HIV-1 Replication in MT-2 Cells

As a first approach to study the mechanism of action of globotriose, an experiment involving one-round of replication was performed. The CD4′ human T lymphotrophic virus (HTLV-1) transformed cell line MT-2 was obtained from the National Institute for Biological Standards and Control, Medical Research Council, UK (repository reference ARP014). The cells were maintained in RPMI-1640 plus 5% heat-inactivated fetal bovine serum (FBS). MT-2 cells were incubated with HIV-1 at low (0.001 pfu/cell) or high (2.5 pfu/cell) multiplicity of infection (MOI) in the presence of different concentrations of drugs for different periods of time. High-titer stocks of HIV-1 pNL4.3 strain (kindly provided by Dr. J. Alcami, Hospital Doce de Octubre, Madrid, Spain) were prepared (Adachi et al., J. Virol. 59:284-291, 1986), and the titers were determined using the endpoint dilution method of Karber (Arch. Exp. Pathol. Pharmakol. 162:480-483, 1931).

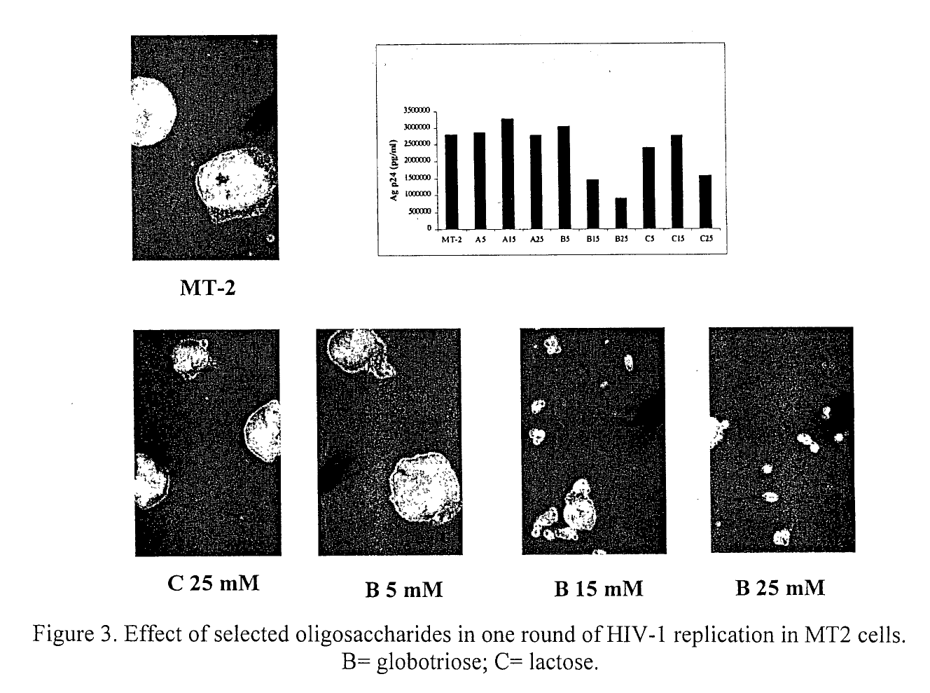

Briefly, 10⁶ MT-2 cells were infected with HIV-1 at a MOI of 0.001 in a final volume of 5 ml of complete medium (RPMI-1640 plus 5% heat-inactivated FBS). After 3-4 days, 75% of the cells had formed syncytia (i.e., cells that had fused into giant multinucleated cells). 30×10⁶ MT-2 cells in 30 ml of complete medium were added to the culture and incubated again until 75% of the cells had formed synctitia in the culture. The cells were then centrifuged at 1200 rpm for 10 minutes; the supernatant was filtered through a 0.4 μm pore filter and titrated. Stocks of the M-tropic strain Ba-L (Gartner et al., Science 233:215-219, 1986) and the T-tropic clinical isolate 1936 (Muñoz-Fernandez et al., Pediatr. Res. 45:597-602, 1996), were similarly prepared using human peripheral blood monocytes and lymphocytes, respectively. The formation of syncytia was analyzed by phase contrast and fluorescence microscopy with 1 μg/ml Hoechst 33258 (Sigma Chemical, Co. St. Louis, Mo.) for 7 minutes on ice after staining to visualize the nuclei (FIG. 3B).

Three different concentrations (0.5-50.0 mM) of the three oligosaccharide compounds, globotriose, lactose and lacto-N-tetraose were used. Globotriose completely inhibited the formation of syncytia, both in terms of number and size of syncytia, at a concentration of 25 mM. At lower concentrations, globotriose still had an effect on the formation of syncytia since the size of these syncytia (i.e., the number of nuclei as an indication of the number of fused cells) was decreased. Lactose had an effect although weaker than that exhibited by globotriose. Since syncytia are formed after fusion of HIV-1-infected cells that express the envelope protein on the cell surface with non-infected CD4⁺ cells, the results, as shown in FIG. 3, indicate that oligosaccharides directly prevent HIV fusion with target cell membranes, therefore preventing the formation of syncytia.

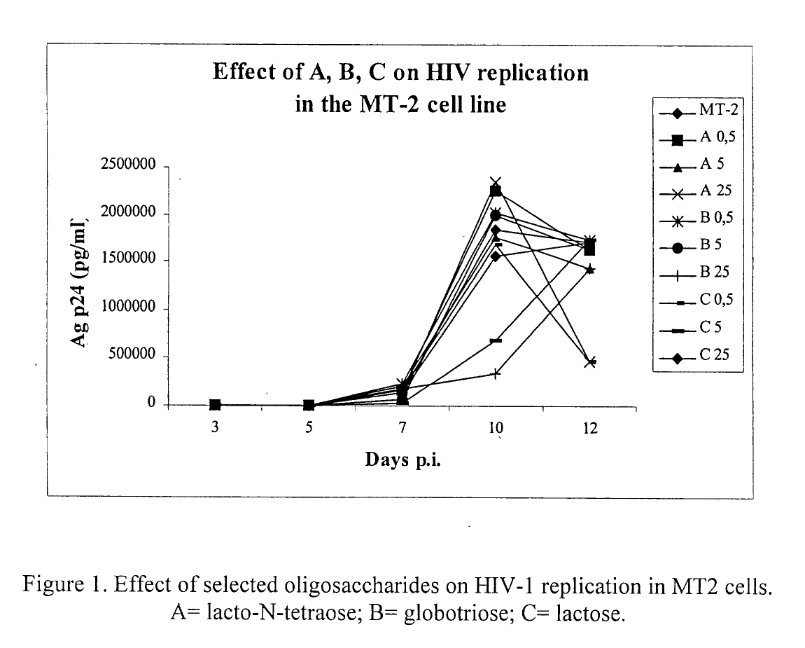

Additionally, viral replication was monitored each 3-4 days by measuring release of viral p24 antigen in the supernatant of the cultures using an antigen capture immunoassay. The release of antigen p24 was inhibited by globotriose at a concentration of 25 mM but not at 5 mM or lower. The inhibitory effect was maximum at days 3 and 7, but it was recovered at day 10 postinfection. Lactose had an effect although less potent than globotriose. Lacto-N-tetraose was ineffective (See FIG. 1).

Toxicity of high concentrations of oligosaccharides was assessed by counting MT-2 cells (100,000 cells/ml) after incubation with globotriose and lactose at concentrations ranging from 0.5-50 mM. After 2, 4 and 7 days, the number of cells was counted in a Neubauer chamber (BRAND, Germany) and their viability was estimated by Tripan Blue exclusion. At a concentration of 50 mM, globotriose was the only treatment to cause cell toxicity. Globotriose at 50 mM inhibited the proliferation of MT-2 cells and decreased their viability to 60% after 4 days of culture (data not shown). Lactose at concentrations as high as 50 mM did not cause significant toxicity. These results indicate that globotriose may be toxic at this high concentration. Lower concentrations of globotriose (including 25 mM) showed no toxic effect on MT-2 cells.

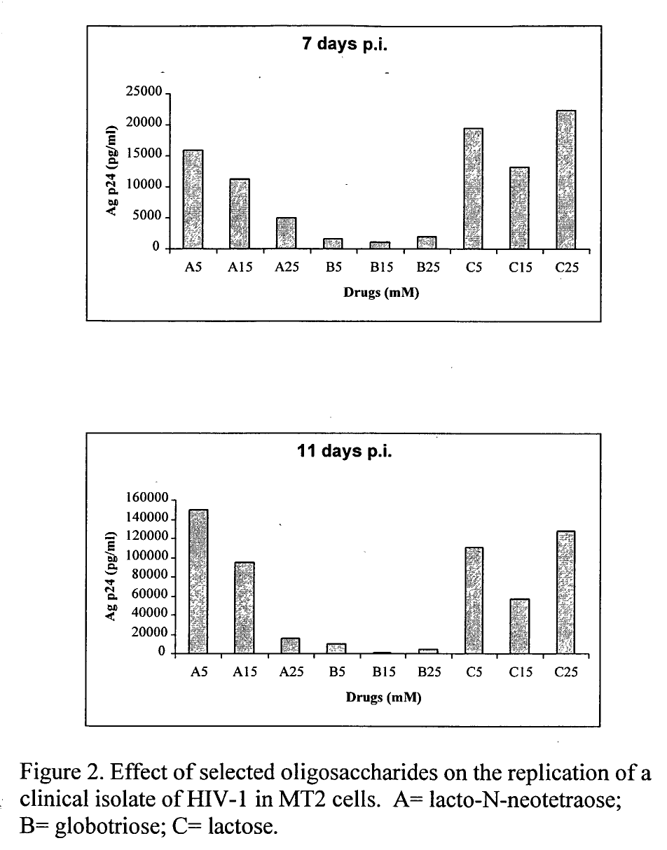

The effect of the oligosaccharides was also tested on the replication of a clinical isolate 1936 of HIV-1 (T-tropic). MT-2 cells were infected as described above, and the course of infection was followed by the release of p24 and the formation of syncytia. As shown in FIG. 2, globotriose (B) inhibited replication of this strain of HIV-1 at all concentrations, at days 7 and 11, suggesting that it may have a broad spectrum of activity against clinically relevant HIV-1 strains.

### EXAMPLE 2

### Effect of Oligosaccharides on CD4 Expression

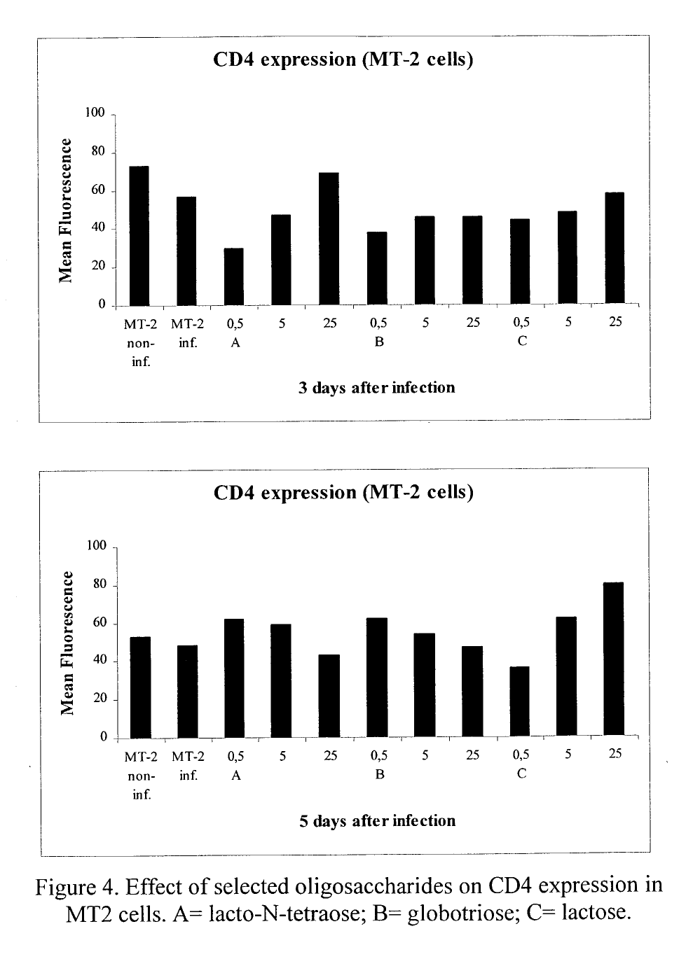

Some glycosphingolipids inhibit HIV-1 replication by inducing the down-regulation of CD4 receptors on HIV-1 sensitive cells. To determine whether CD4 expression was altered in the presence of the oligosaccharides, HIV-1 infected cells treated with the oligosaccharides of the present invention were washed in PBS (Phosphate Buffered Saline) and incubated on ice with FITC-conjugated anti CD4 antibody Leu3a (BD Biosciences, Heidelberg, Germany) for 30 minutes. The samples were washed, fixed with 1% paraformaldehyde and analyzed by flow cytometry. As shown in FIG. 4, expression of CD4 receptors remained at similar levels independent of the dose and of the compound used in the experiment. These results demonstrate that the antiviral activity of the oligosaccharides is not mediated by a down regulation or masking of the CD4 receptors, but rather by preventing HIV-1 fusion with the target cell.

### EXAMPLE 3

### Effect of Oligosaccharides on HIV-1 Replication in Monocytes

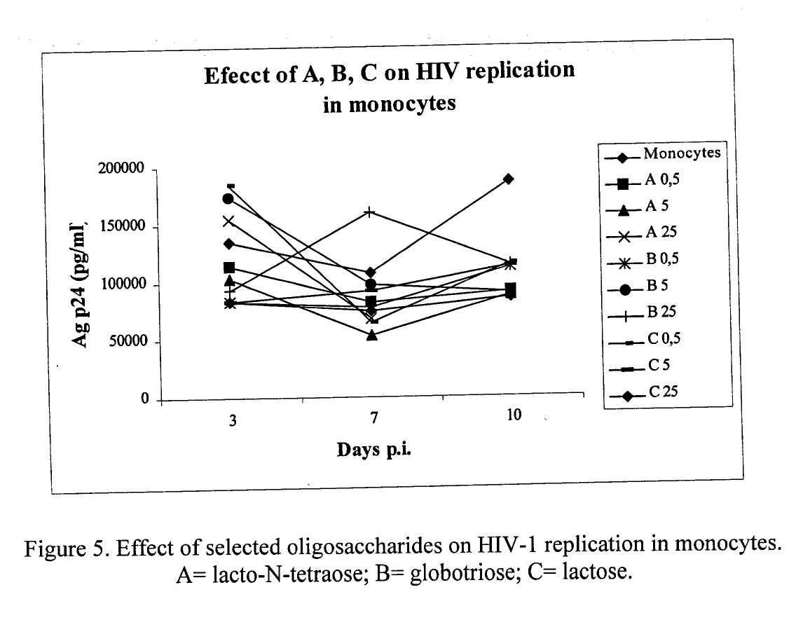

Human peripheral blood monocytes were isolated from whole blood of healthy donors by Ficoll Hypaque density gradient centrifugation (Pharmacia Fine Chemicals, Uppsala, Sweden). The mononuclear cell fraction was incubated in culture dishes for 24 hours at 37° C. Non-adherent cells were discarded, and adherent cells were maintained in RPMI medium plus 5% fetal bovine serum for 6 days. The cells were determined to be more than 80% positive for the monocytic marker CD14 by flow cytometry. Adherent monocytes were subsequently infected at a MOI of 0.001 with BAL strain (M-tropic) of HIV-l in the presence of globotriose (B), lactose (C) and lacto-N-tetraose (A) at concentrations of 0.5, 5 and 25 mM. Viral antigen p24 release was measured as an estimate of viral replication. Unlike its effects on the replication of the T-tropic strain of HIV-1, pNL4.3, globotriose did not inhibit the replication of the R5 M-tropic strain Ba-L in cultured human peripheral blood monocytes. Lactose and lacto-N-tetraose were also ineffective (FIG. 5). These findings emphasize the potential usefulness of oligosaccharides in treating or preventing infection by syncytia-forming types of viruses.

### EXAMPLE 4

### Synergistic Effects of Lactose and Globotriose

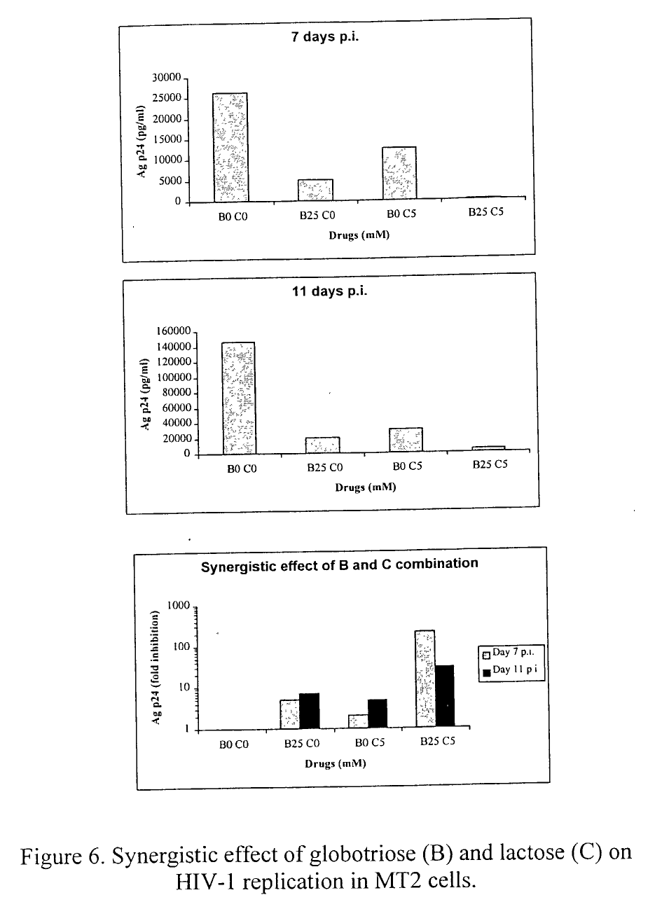

Based on the anti-HIV-1 activity of both globotriose and lactose, the effect of their combination was assessed by following the procedures indicated in Example 1. The combination of 25 mM globotriose and 5 mM lactose exerted a synergistic effect. At day 7 post-infection, 25 mM globotriose reduced by 5 fold the production of p24, and 5 mM lactose reduced it by 2 fold. The combination of both oligosaccharides at the same concentrations completely inhibited viral replication reducing p24 release by 222 fold (FIG. 6). This unexpected result represents an advantage over other therapies already in existence. The possibility of using both oligosaccharides simultaneously results in an enhanced therapeutic activity with no collateral toxic effects as may be present using other therapeutic approaches.

In conclusion, these examples demonstrate that the monovalent oligosaccharides of the present invention were active against the laboratory strain pNL4.3 of HIV-1 and also against a clinical isolate of a T-tropic strain of HIV-1 at non-toxic concentrations.

Additionally, the examples indicate that the monovalent oligosaccharides of the present invention can be used alone or in combination. The combination of low concentrations of the two oligosaccharides of the present invention results in a greater anti-viral activity than that observed upon using either oligosaccharide alone, without increasing potential collateral effects that could result by increasing the concentration of each one individually.

These results also suggest that the monovalent oligosaccharides of the present invention may have a broad spectrum of activity not only against clinically relevant HIV-1 strains, but also against other syncytia-forming viruses.

In conclusion, the effects of the monovalent oligosaccharides of the present invention can be used alone or in combination not only as a therapeutic tool to treat established infections, but may also be used as a preventive treatment in individuals at risk for contracting HIV-1 or other syncytia-forming viruses.

1. A method of inhibiting the fusion of a retrovirus with cell membranes, comprising the step of administering a composition comprising at least one monovalent oligosaccharide to a mammal in an amount sufficient to effect said inhibition of fusion.

2. A method of inhibiting retrovirus-mediated syncytia formation, comprising the step of administering a composition comprising at least one monovalent oligosaccharide to a mammal in an amount sufficient to effect said inhibition of retrovirus-mediated synctitia formation.

3. The method of claim 1 or 2, wherein the retrovirus is a Human Immunodeficiency Virus (HIV).

4. The method of claim 3 wherein the HIV is Human Immunodeficiency Virus type 1 (HIV-1).

5. The method of claim 1 or 2, wherein the retrovirus is a syncytia-forming virus.

6. The method of claim 5 wherein the syncytia-forming virus is a HIV-1 variant.

7. The method of claim 1, wherein said composition comprises the combination of at least two of said monovalent oligosaccharides in amount sufficient to synergistically augment said inhibition of fusion.

8. The method of claim 2, wherein said composition comprises the combination of at least two of said monovalent oligosaccharides in amount sufficient to synergistically augment said inhibition of syncytia formation.

9. A method of preventing an infection in a mammal caused by a retrovirus, comprising the step of administering a composition comprising at least one monovalent oligosaccharide to a mammal, wherein said composition is administered in an amount sufficient to effect said prevention.

10. The method of claim 9 wherein said composition comprises the combination of at least two of said monovalent oligosaccharides in amount sufficient to effect said prevention.

11. A method of treating an infection in a mammal caused by a retrovirus, comprising the step of administering a composition comprising at least one monovalent oligosaccharide to a mammal, wherein said composition is administered in an amount sufficient to effect said treatment.

12. The method of claim 11 wherein said composition comprises the combination of at least two of said monovalent oligosaccharides in amount sufficient to effect said treatment.

13. The method of claim 9 or 11 wherein said retrovirus is a Human Immunodeficiency Virus (HIV).

14. The method of claim 13 wherein said HIV is the Human Immunodeficiency Virus type 1 (HIV-1).

15. A method for preventing transmission of HIV in a mammal, comprising the step of administering a composition comprising at least one monovalent oligosaccharide to said mammal, wherein said composition is administered in an amount sufficient to prevent said transmission.

16. The method of claim 15 wherein said composition comprises the combination of at least two of said monovalent oligosaccharides in an amount sufficient to effect said prevention of said transmission.

17. The method of claim 15, wherein the transmission is perinatal vertical transmission.

18. A composition comprising at least one monovalent oligosaccharide, wherein said at least one monovalent oligosaccharide inhibits interaction of CD4 receptors, viral gp120 and membrane glycolipids.

19. The composition of claim 18 wherein said at least one monovalent oligosaccharide is selected from the group consisting of globotriose and lactose.

20. The composition of claim 19 wherein said at least one monovalent oligosaccharide is globotriose.

21. The composition of claim 20 wherein said globotriose is present in a concentration between 5 mM and 25 mM.

22. The composition of claim 19 wherein said at least one monovalent oligosaccharide is lactose.

23. The composition of claim 22 wherein said lactose is present in a concentration between 5 mM and 25 mM.

24. The composition of claim 18 wherein said composition is selected from the group consisting of a pharmaceutical composition and a nutritional composition.

25. The composition of claim 24 wherein said composition can be administered by a route selected from the group consisting of parenteral administration, enteral administration, and dermal administration.

26. The composition of claim 25 wherein said parenteral administration is intravenous.

27. The composition of claim 25 wherein said enteral administration is oral.

28. The composition of claim 25 wherein said dermal administration is local.
